(19) United States
(12) Patent Application Publication
(10) Pub. No.: US 2020/0014863 A1
(43) Pub. Date: Jan. 9, 2020

(54) IMAGE CAPTURING APPARATUS

(71) Applicant: SHARP KABUSHIKI KAISHA, Sakai City (JP)

(72) Inventors: MASAHIRO SHIOTA, Sakai City (JP); SHIGENARI TAGUCHI, Sakai City (JP); KUNIHIKO IIZUKA, Sakai City (JP); NOBUYUKI ASHIDA, Sakai City (JP)

(21) Appl. No.: 16/503,201

(22) Filed: Jul. 3, 2019

Related U.S. Application Data
(60) Provisional application No. 62/695,355, filed on Jul. 9, 2018.

(51) Int. Cl. H04N 5/343; G03B 42/02; A61B 6/00; H04N 5/347; H04N 5/345; H04N 5/365; H04N 5/32; G01T 1/24

(52) U.S. Cl. CPC H04N 5/343; G03B 42/02; A61B 6/4233; G01T 1/24; H04N 5/345; H04N 5/3651; H04N 5/32; H04N 5/347

(57) ABSTRACT

[Object] To realize an image capturing apparatus capable of suppressing noise in an image.

[Solution] An image capturing apparatus (100) includes a plurality of pixels (110) arranged in a matrix form. Each pixel includes a switching element (112) configured to control outputting of a charge accumulated in a sensor element (111). Pixels in each column are grouped into blocks (120). Each block includes a block switching element (122) configured to control outputting.

### TECHNICAL FIELD

The present invention relates to an image capturing apparatus, and more particularly, to an X-ray image capturing apparatus using a planar detector.

### BACKGROUND ART

As for a sensor element configured to output an electric signal corresponding to a dose of incident radiation, in particular, of an X-ray, a direct conversion type sensor element configured to directly convert an X-ray to an electric signal, and an indirect conversion type sensor element configured to convert an X-ray to light using a scintillator and then further to an electric signal using a photoelectric conversion element are used. As disclosed in PTL 1, a panel for capturing an X-ray image has been developed, in which a plurality of pixels each using an X-ray sensor element such as that described above are arranged in a two-dimensional matrix form on a substrate.

In this panel, a gate terminal of a TFT (Thin Film Transistor) functioning as a switching element disposed in each of pixels of each row of the two-dimensional matrix is connected in common to a row selection line (a gate drive line) on a row-by-row basis. Each row selection line is connected to a line output terminal of a gate driver. A drain terminal of a TFT of each pixel is connected in common to an output line (a data signal line) on a column-by-column basis in the two-dimensional matrix. Each output line is connected to corresponding one of line input terminals of a multiplexer via a readout amplifier and a readout circuit realized using an integration circuit including a time-constant capacitor and a reset switch.

### CITATION LIST

### Patent Literature

[PTL 1] Japanese Unexamined Patent Application Publication No. 2010-148673 (laid open on Jul. 8, 2010)

### SUMMARY OF INVENTION

### Technical Problem

In the image capturing apparatus of the above-described type, there is a need for a higher-definition and larger-sized apparatus to expand applications and improve performance. To this end, it is necessary to greatly increase the number of pixels. For example, in a case where there are 1000×1000 pixels, 1000 switching elements are connected in parallel to one readout circuit. As a result, a great increase occurs in capacitance, seen from the readout circuit to the output line side (the input side of the readout circuit), originating from switching elements. Such capacitance of the output line has an influence on thermal noise on the output line, which is one of main factors causing degradation in performance of an X-ray image panel. Therefore, the grater the number of pixels, the greater the thermal noise on the output line occurs and the greater the degradation in the performance (SN ratio) of the X-ray image panel occurs. This can be a problem to be solved to achieve a large-sized X-ray image panel.

It is an object of an aspect of the present invention to realize an image capturing apparatus capable of suppressing noise in an image.

### Solution to Problem

(1) In an aspect of the present invention, an image capturing apparatus includes a plurality of pixels arranged in a matrix form, each pixel including a sensor element configured to accumulate a charge corresponding to an amount of a radiation and a switching element configured to control outputting of the charge accumulated in the sensor element, wherein pixels belonging to each same column are grouped into blocks each including a plurality of pixels, and each block includes a block switching element configured to control outputting of a charge output from the switching elements located in the block.

(2) In an aspect of the present invention based on (1), the image capturing apparatus further includes a row selection circuit configured to sequentially select pixels in each row, wherein the block switching element in the block is controlled such that the block switching element is in an on-state in a period in which a row, to which a pixel in the block belongs, is selected, and the block switching element is in an off-state in a period in which a row other than a row to which any pixel in the block belongs is selected.

(3) In an aspect of the present invention based on (1) or (2), in the image capturing apparatus, the block includes a block output line to which a charge is output from each switching element located in the block.

(4) In an aspect of the present invention based on (3), in the image capturing apparatus, pixels in the block are disposed on both sides of the block output line.

(5) In an aspect of the present invention based on one of (1) to (4), the image capturing apparatus further includes a readout circuit configured to convert an amount of charge output from the block switching element to an electric signal.

### Advantageous Effects of Invention

According to an aspect, the present invention provides an effect that an image capturing apparatus capable of suppressing noise in an image is achieved.

### BRIEF DESCRIPTION OF DRAWINGS

FIG. 1 is a diagram illustrating a circuit configuration of an image capturing apparatus according to a first embodiment of the present invention.

FIG. 2 is a schematic diagram illustrating a layout of pixels of the image capturing apparatus according to the first embodiment of the present invention.

FIG. 3 is a timing chart illustrating an operation of the image capturing apparatus according to the first embodiment of the present invention.

FIG. 4 is a schematic diagram illustrating a layout of pixels of an image capturing apparatus according to a second embodiment of the present invention.

### DESCRIPTION OF EMBODIMENTS

Embodiments of the present invention are described below with reference to drawings. Note that in these embodiments, materials, shapes, and relative positions of parts and methods of processing parts are described by way of example but not limitation. That is, the invention is not limited to the details of these examples described in the embodiments. Furthermore, the drawings are schematic, and scaling ratios and shapes may be different from actual ones. In the drawings, the same or similar constituent elements may be denoted by the same reference symbols.

### First Embodiment

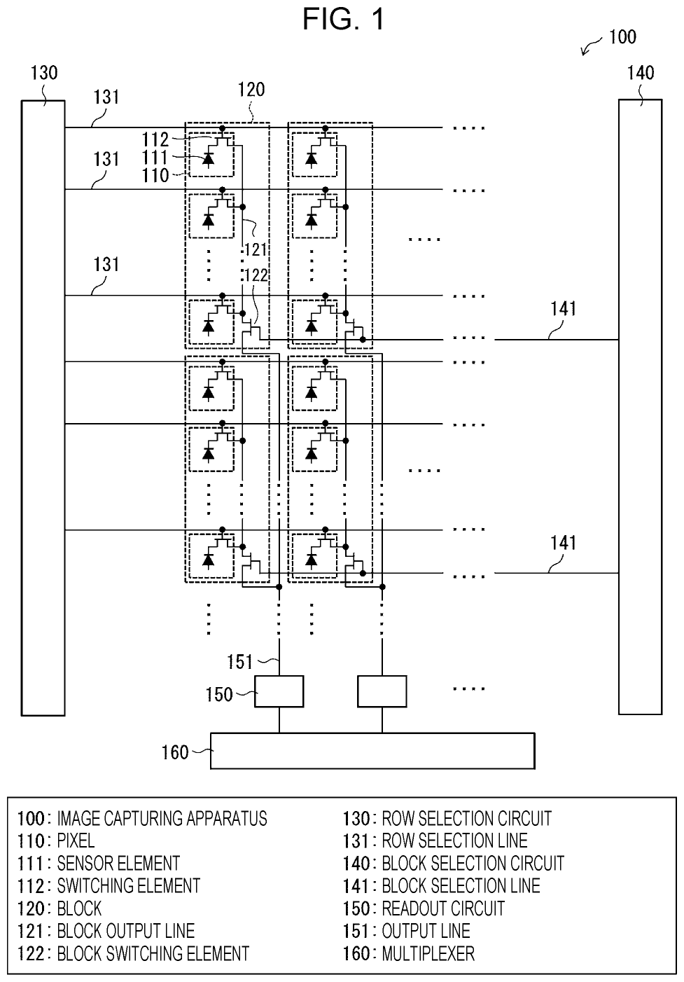

A first embodiment of the present invention is described with referent to FIG. 1 to FIG. 3.

(Configuration of Image Capturing Apparatus 100)

FIG. 1 is a diagram illustrating a circuit configuration of an image capturing apparatus 100 of the present embodiment. As shown in the figure, an image capturing apparatus 100 includes a plurality of pixels 110 arranged in a matrix form. Each pixel 110 includes a sensor element 111 and a switching element 112. The switching element 112 is a switch that controls outputting from the pixel 110 to the outside. A source terminal of the switching element 112 is connected to one of terminals of the sensor element 111. In the present embodiment, a known switching element such as a FET using a TFT or the like may be used as the switching element 112.

Gate terminals of respective switching elements 112 of pixels 110 in each same row are connected, via a common row selection line 131, to one of the output terminals of a first gate driver functioning as a row selection circuit 130. In the present description, as described above, pixels in the same row are given by pixels 110 connected to one common row selection line 131. In the present embodiment, the number of rows may be 1000.

Pixels 110 in each same column are grouped into blocks 120 including a plurality of pixels. Each block 120 includes a block output line 121 and a block switching element 122. The block output line 121 is connected to drain terminals of switching elements 112 of respective pixels 110 in the block to which the block output line 121 belongs. The block switching element 122 is a switch that controls outputting from the block 120 to the outside, and a source terminal thereof is connected to an end of the block output line 121.

In the present embodiment, a TFT is used as the block switching element 122. However, a known switching element such as a FET may be used as the block switching element 122. Furthermore, in the present embodiment, each block 120 includes 100 pixels 110. Thus, the number of rows (block rows) formed by the blocks 120 is 10.

Gate terminals of block switching elements 122 of blocks 120 belonging to one same block row are connected, via a common block selection line 141, to one of output terminals of a second gate driver functioning as a block selection circuit 140.

Drain terminals of respective block switching elements 122 in blocks 120 in one same column are connected, via a common output line 151, to an input terminal of corresponding one of readout circuits 150 provided for the respective columns. Outputs from the respective readout circuits 150 are input to a multiplexer 160. In the present description, as described above, pixels in one same column are given by pixels 110 connected to one common output line 151. In the present embodiment, the number of columns may be 1000. A known readout circuit configured to read an accumulated charge may be employed as the readout circuit 150. For example, an integration circuit similar to that disclosed in PTL 1 may be employed.

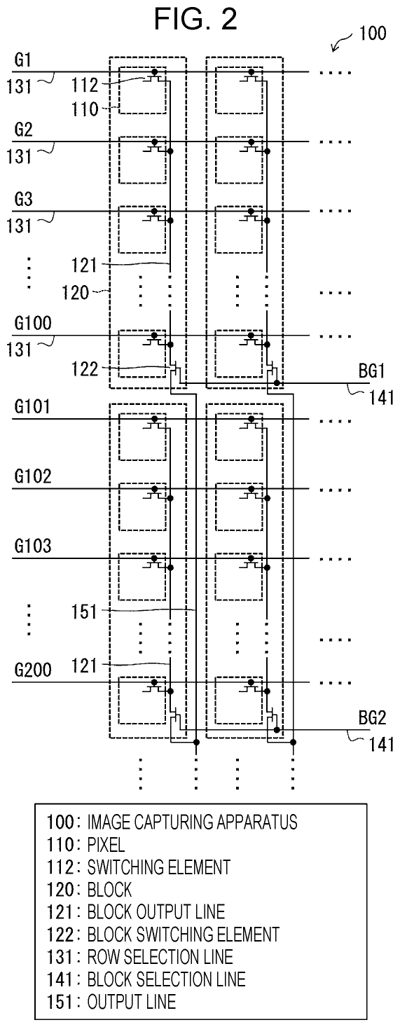

FIG. 2 is a diagram schematically illustrating a layout of pixels in the image capturing apparatus 100 according to the present embodiment. In FIG. 2, each pixel 110 is schematically represented by a rectangular frame, and each switching element 112 is represented by a circuit symbol. Furthermore, in FIG. 2, a row selection line 131 in an n-th row is denoted by a symbol Gn, and a block selection line 141 in an n-th block-row is denoted by a symbol BGn.

In the present embodiment, one row selection line 131 is provided in each row in a geometrical arrangement of pixels 110, and one output line 151 is provided in each column in the geometrical arrangement of pixels 110. Therefore, in the circuit configuration including 1000 rows and 1000 columns according to the present embodiment, the geometrical array also has 1000 rows and 1000 columns.

(Operation of Image Capturing Apparatus 100)

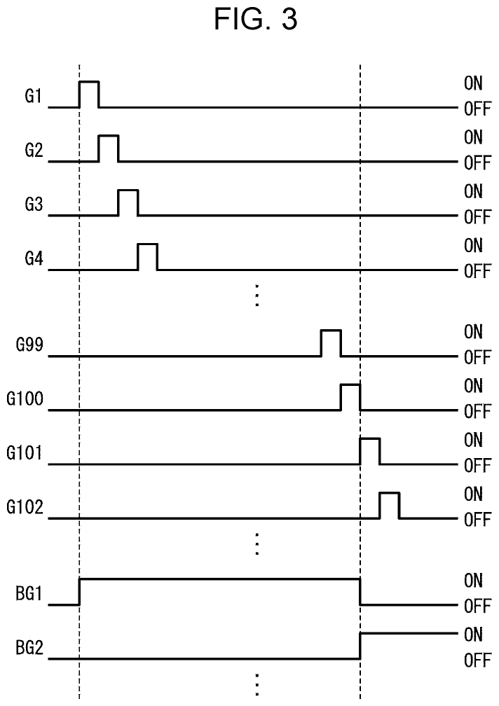

Next, an operation of the image capturing apparatus 100 according to the present embodiment is described referring to a timing chart shown in FIG. 3.

The row selection circuit 130 outputs a row selection signal to row selection lines G1 to G1000. As shown in FIG. 3, the row selection lines G1 to G1000 are sequentially selected such that an on-signal is output to only one row selection line Gn at a time, and an off-signal is output to not-selected row selection lines Gn.

The block selection circuit 140 outputs a block selection signal to the block selection lines BG1 to BG10 such that an on-signal is output to a block selection line 141 connected to a selected block 120, while an off-signal is output to non-selected block selection lines 141. A row, to which one of pixels 110 included in the selected block 120 belongs, is selected by the row selection circuit 130.

For example, a block 120 in a 1st block-row includes pixels 110 in 1st to 100th rows, and a block 120 in a 2nd block-row includes pixels 110 in 101st to 20000 rows. During a period in which the row selection circuit 130 selects one of G1 to G100, the block selection circuit 140 selects the block selection line BG1. During a period in which the row selection circuit 130 selects one of G101 to G200, the block selection circuit 140 selects the block selection line BG2. As described above, the block selection circuit 140 sequentially and selectively output the on to the block selection lines BG1 to BG10.

That is, a block switching element 122 in a specific block 120 is in the on-state in a period in which a row, to which a pixel 110 in this specific block 120 belongs, is selected by the row selection circuit 130, but this block switching element 122 is in the off-state in a period in which a row other than a row to which any pixel 110 in the block 120 belongs is selected. Thus, the pixels 110 in the specific block 120 are disconnected by the block switching element 122 from the output line 151 over the period in which any row other than a row to which any pixel 110 in the block 120 belongs is selected.

By controlling the switching element 112 and the block switching element 122 in the above-described manner, pixels 110 are read out as follows.

A switching element 112 of a pixel 110 belonging to a selected row is turned on by an on-signal applied to a gate. The sensor element 111 of this pixel 110 is electrically connected to the block output line 121 of the block 120 including this pixel 110. The block switching element 22 of the present block 120 is turned on by an on-signal applied to a gate via the block selection line 141, and this block output line 121 is electrically connected to the output line 151.

As a result, a charge accumulated in the sensor element 111 is transferred to a readout circuit 150 via the switching element 112, the block output line 121, the block switching element 122, and the output line 151. The transferred charge is converted to a specific electric signal corresponding to the amount of the accumulated charge and is input to the multiplexer 160.

When the row-selection period for the pixel 110 is ended, the switching element 112 turns off. As a result, the sensor element 111 of the pixel 110 is electrically disconnected from the output line 151, and a charge corresponding to the amount of incident X-ray (radiation) is accumulated until the pixel 110 is again selected.

Thus, in each column, amounts of X-ray incident to respective pixels 110 are converted to electric signals and read out via the sequential row selection. As described above, pixels in each column are read out in a time-sequentially and two-dimensional X-ray image data is obtained as is obtained by the technique disclosed in PTL 1.

(Effects of Image Capturing Apparatus 100)

For comparison, X-ray image data was obtained (as a comparative example) using an image capturing apparatus having a circuit configuration with a circuit complexity similar to that according to the present embodiment, that is having 1000 rows by 1000 columns based on a conventional technique. Herein, the conventional technique refers to a technique in which pixels in each column are not grouped into blocks. The image obtained by the image capturing apparatus in the comparative example included significant noise, and contrast of the image was low, that is, the image quality was clearly lower than is obtained by the image capturing apparatus 100 according to the present embodiment.

The difference described above occurs for following reasons. The switching elements and the block switching elements each have gate-drain capacitance Cs. Therefore, in the image capturing apparatus using the conventional technique, the capacitance (the capacitance of the output line) seen to the input side from the readout circuit in each column is proportional to the number of pixels (switching elements) connected to the column. In a case where the circuit complexity of the image capturing apparatus is not large, that is, in a case where the number of rows is about 100, the capacitance of the output line is equal to about 100 Cs. In contrast, when the number of rows is 1000, the capacitance of the output line is equal to about 1000 Cs. That a great increase in capacitance occurs as a result of the scale-up of the number of pixels.

The capacitance of the output line is related to a level of noise that occurs when the amount of accumulated charge is read by the readout circuit 150. Thus, in the conventional technique, the above-described scale-up of the number of pixels results in degradation in image quality. In the case of the comparative example having 1000 rows, noise of an image caused by the ate-drain capacitance Cs is about 10 times greater than that of a small-scale image capturing apparatus having about 100 rows.

In contrast, in the image capturing apparatus 100 according to the present embodiment, the capacitance (the capacitance of the output line) seen to the input side from the readout circuit 150 in each column is given by a parallel connection, in a certain period, of Cs of switching elements 112 of pixels 110 in a selected block (the block switching element of which is in the ON-state) and Cs of block switching elements 122 of non-selected blocks 120. The capacitance of the output line is equal to about 109 Cs, which is similar to that of a small-scale image capturing apparatus having about 100 rows. Thus, in the image capturing apparatus 100 according to the present embodiment, it is possible to achieve an image capturing apparatus including a large number of pixels and capable of providing high image quality, compared to the quality provided by the comparative example of the image capturing apparatus, without substantial degradation in image due to the scale-up.

### Second Embodiment

An image capturing apparatus according to a second embodiment of the present invention is described below. The present embodiment is different in circuit configuration from the image capturing apparatus 100 according to the first embodiment.

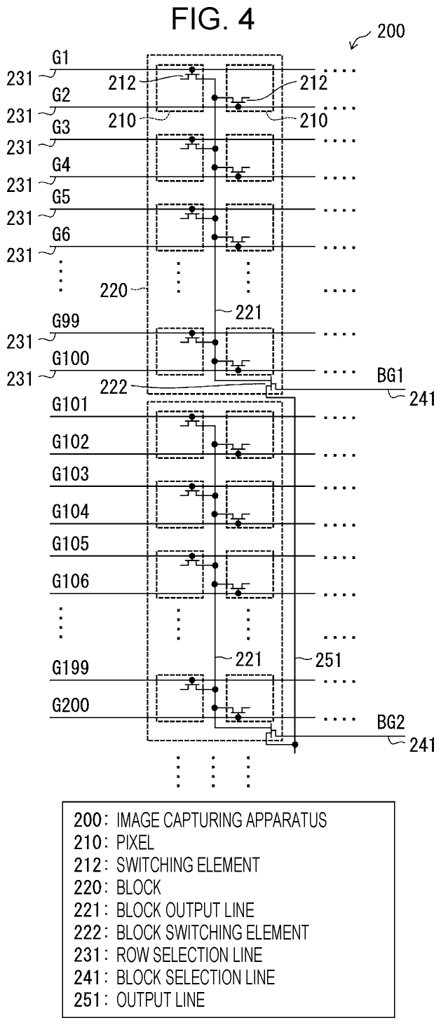

FIG. 4 is a diagram schematically illustrating a layout of pixels in the image capturing apparatus 200 according to the present embodiment. In FIG. 4, each pixel 210 is schematically represented by a rectangular frame, and each switching element 212 is represented by a circuit symbol. As in FIG. 2, sensor elements are not shown. Furthermore, a row selection line 231 in an n-th row is denoted by Gn, and a block selection line 241 in an n-th block-row is denoted by a symbol BGn.

In the image capturing apparatus 200 according to the present embodiment, unlike the image capturing apparatus 100 according to the first embodiment, one output line 251 is provided for every two columns in a geometrical arrangement of pixels 210. In each block 220, pixels 210 are disposed on right-hand and left-hand sides of the block output line 221, and are connected to a common block output line 221. The block output line 221 is connected to an output line 251 via a block switching element 222. The output line 251 is disposed on one side (the right-hand side) of the block 220. The pixels 210 on the right-hand and left-hand sides are connected to the same output line 251 and thus they belong to the same column. That is, in the present embodiment, columns in the geometrical arrangement of pixels are not identical to columns defined in the circuit configuration.

In the block 220, each two adjacent pixels 210 located on right-hand and left-hand sides of the block output line 221 are located in a geometrically same row, but they are connected to different row selection lines 231 (for example, G1 and G2). That is, rows in the geometrical arrangement are not identical to rows defined in the circuit configuration.

In the present embodiment, the geometrical pixel array includes 1000 rows and 1000 columns. However, the pixel array defined in the circuit configuration includes 2000 rows and 500 columns. The number of columns is 500, and thus correspondingly, 500 readout circuits are necessary. Compared to the image capturing apparatus 100 according to the first embodiment, although the geometric array of pixels is the same, the number of necessary readout circuits can be reduced to half.

Furthermore, in the present embodiment, the block output line 221 and the output line 251 are disposed in the above-described manner, and thus only one of the block output line 221 and the output line 251 is disposed between adjacent right-hand and left-hand pixels 210 (except for the block 220 in the top row). Thus, it is possible to dispose pixels 210 more densely than is possible in the image capturing apparatus 100 according to the first embodiment in which both the block output line 121 and the output line 151 are disposed between adjacent right-hand and left-hand pixels 210.

Also in the present embodiment, as in the first embodiment, pixels 210 connected to each same output line 251 (pixels 210 in each same column) are grouped into a plurality of blocks 220. As shown in FIG. 4, the number of pixels disposed in each block 220 may be, for example, 100. In this case, the number of blocks disposed in each column is 20, and the total number of block selection lines 241 is 20.

(Operation of Image Capturing Apparatus 200)

Also in the present embodiment, the operation may be similar to that of the image capturing apparatus 100 according to the first embodiment. That is, although there are differences in the total number of rows and the total number of block-rows, signals can be sequentially read out from the pixels 210 via the operation described in the time chart shown in FIG. 3.

(Effects of Image Capturing Apparatus 200)

In the image capturing apparatus 200 according to the present embodiment, the capacitance (the capacitance of the output line) seen to the input side from the readout circuit in each column is given by a parallel connection, in a certain period, of capacitance Cs of switching elements 212 in a selected block 220 (whose block switching element 222 is in the on-state) and capacitance Cs of switching elements 222 in a non-selected block 220. Therefore, it is about 119 Cs which is similar to the capacitance of a small-scale image capturing apparatus having about 100 rows. Thus, in the image capturing apparatus 200 according to the present embodiment, it is possible to achieve an image capturing apparatus including a large number of pixels and having substantially no degradation in image due to the scale-up.

The present invention is not limited to the embodiments described above, but various modifications are possible within the scope described in claims. Embodiments may also be possible by properly combining technical means disclosed in different embodiments, and the resultant embodiments fall within the technical scope of the present invention. Furthermore, by combining technical means disclosed in embodiments, it is possible to create a new technical feature.

### REFERENCE SIGNS LIST

100, 200 image capturing apparatus

110, 210 pixel

111 sensor element

112, 212 switching element

120, 220 block

121, 221 block output line

122, 222 block switching element

130 row selection circuit

131, 231 row selection line

140 block selection circuit.

141, 241 block selection line

150 readout circuit

151, 251 output line

160 multiplexer

1. An image capturing apparatus comprising:
a plurality of pixels arranged in a matrix form, each pixel including a sensor element configured to accumulate a charge corresponding to an amount of a radiation and a switching element configured to control outputting of the charge accumulated in the sensor element,
wherein pixels belonging to each column are grouped into blocks each including a plurality of pixels, and
each block includes a block switching element configured to control outputting of a charge output from the switching elements located in the block.

2. The image capturing apparatus according to claim 1, further comprising
a row selection circuit configured to sequentially select pixels in each row,
wherein the block switching element in the block is controlled such that
the block switching element is in an on-state in a period in which a row, to which a pixel in the block belongs, is selected, and
the block switching element is in an off-state in a period in which a row other than a row to which any pixel in the block belongs is selected.

3. The image capturing apparatus according to claim 1, wherein the block includes a block output line to which a charge is output from each switching element located in the block.

4. The image capturing apparatus according to claim 3, wherein pixels in the block are disposed on both sides of the block output line.

5. The image capturing apparatus according to claim 2, wherein the block includes a block output line to which a charge is output from each switching element located in the block.

6. The image capturing apparatus according to claim 5, wherein pixels in the block are disposed on both sides of the block output line.

7. The image capturing apparatus according to claim 1, further comprising a readout circuit configured to convert as amount of charge output from the block switching element to an electric signal.
